(19) United States
(12) Patent Application Publication
(10) Pub. No.: US 2020/0108132 A1
(43) Pub. Date: Apr. 9, 2020

(54) ANTI-MALARIA COMPOSITIONS AND METHODS

(71) Applicant: Artificial Cell Technologies, Inc., New Haven, CT (US)

(72) Inventor: Thomas J. Powell, Madison, CT (US)

(21) Appl. No.: 16/589,540

(22) Filed: Oct. 1, 2019

Related U.S. Application Data
(60) Provisional application No. 62/741,198, filed on Oct. 4, 2018.

(51) Int. Cl. A61K 39/015; A61K 9/16; A61P 33/06

(52) U.S. Cl. CPC A61K 39/015; A61P 33/06; A61K 9/1676

(57) ABSTRACT

Multilayer films comprise polypeptide epitopes from Plasmodium falciparum, specifically a circumsporozoite CIS43 epitope and one or more of circumsporozoite T1, B or T* epitope. The multilayer films are capable of eliciting an immune response in a host upon administration to the host. The multilayer films can include at least one designed peptide that includes one or more polypeptide epitopes from a Plasmodium protozoan.

### CROSS-REFERENCE TO RELATED APPLICATIONS

This application claims priority to U.S. Provisional Application 62/741,198 filed on Oct. 4, 2018, which is incorporated herein by reference in its entirety.

### FIELD OF THE DISCLOSURE

The present disclosure relates to compositions and methods for the prevention of malaria infections, specifically multilayer film compositions containing antigenic epitopes.

### BACKGROUND

Malaria is one of the most prevalent infections in tropical and subtropical areas throughout the world. Malaria infections lead to severe illnesses in hundreds of millions of individuals worldwide, leading to death in millions of individuals, primarily in developing and emerging countries every year. The widespread occurrence and elevated incidence of malaria are a consequence of the increasing numbers of drug-resistant parasites and insecticide-resistant parasite vectors. Other factors include environmental and climatic changes, civil disturbances, and increased mobility of populations.

Malaria is caused by the mosquito-borne hematoprotozoan parasites belonging to the genus Plasmodium. Four species of Plasmodium protozoa (P. falciparum, P. vivax, P. ovale and P. malariae) are responsible for the disease in man; many others cause disease in animals, such as P. yoelii and P. berghei in mice. P. falciparum accounts for the majority of infections and is the most lethal type, sometimes called “tropical malaria”. Malaria parasites have a life cycle consisting of several stages. Each stage is able to induce specific immune responses directed against the corresponding occurring stage-specific antigens.

There is a need for improved antigenic compositions suitable for stimulating an immune response to malaria.

### SUMMARY

In one aspect, a composition comprises

a multilayer film comprising a plurality of alternating oppositely charged polyelectrolyte layers,

wherein one of the polyelectrolyte layers in the multilayer film comprises an antigenic polyelectrolyte, wherein the antigenic polyelectrolyte comprises a Plasmodium falciparum circumsporozoite CIS43 epitope and one or more of Plasmodium falciparum circumsporozoite T1, B, T* and T*^{M} epitopes covalently linked to the antigenic polyelectrolyte;

wherein the polyelectrolytes in the multilayer film comprise a polycationic material or a polyanionic material having a molecular weight of greater than 1,000 and at least 5 charges per molecule.

In one aspect, a composition comprises

a multilayer film comprising a plurality of alternating oppositely charged polyelectrolyte layers,

wherein a first polyelectrolyte layer in the multilayer film comprises a first antigenic polyelectrolyte comprising a covalently linked Plasmodium falciparum circumsporozoite CIS43 epitope, and

wherein a second polyelectrolyte layer in the multilayer film comprises a second antigenic polyelectrolyte comprising one or more of Plasmodium falciparum circumsporozoite T1, B, T* and T*^{M} epitopes covalently linked to the second polyelectrolyte, wherein the first and second polyelectrolyte layers are in the same or different layers,

wherein the polyelectrolytes in the multilayer film comprise a polycationic material or a polyanionic material having a molecular weight of greater than 1,000 and at least 5 charges per molecule.

In another embodiment, a composition comprises

a first multilayer film comprising a plurality of alternating oppositely charged polyelectrolyte layers, wherein a first polyelectrolyte layer in the first multilayer film comprises a first antigenic polyelectrolyte comprising a covalently linked Plasmodium falciparum circumsporozoite CIS43 epitope, and

a second multilayer film comprising a plurality of alternating oppositely charged polyelectrolyte layers, wherein a second polyelectrolyte layer in the second multilayer film comprises a second antigenic polyelectrolyte comprising one or more of Plasmodium falciparum circumsporozoite T1, B, T* and T*^{M} epitopes covalently linked to the second polyelectrolyte,

wherein the polyelectrolytes in the multilayer film comprise a polycationic material or a polyanionic material having a molecular weight of greater than 1,000 and at least 5 charges per molecule.

### BRIEF DESCRIPTION OF THE DRAWINGS

FIG. 1 shows the amino acid sequence of full-length P. falciparum CS protein highlighting specific residues of CIS43 (boxed), T1 (bold), B (bold italics) and T* (bold underlined).

FIG. 2 shows the epitopes and antigenic peptides for circumsporozoite peptide-containing nanoparticles or microparticles. (Top panel) Diagram of P. falciparum CS protein showing locations and sequences of CIS43, T1, B, and T* epitopes. (Bottom panel) Design of CS subunit peptides fused to a poly-lysine tail.

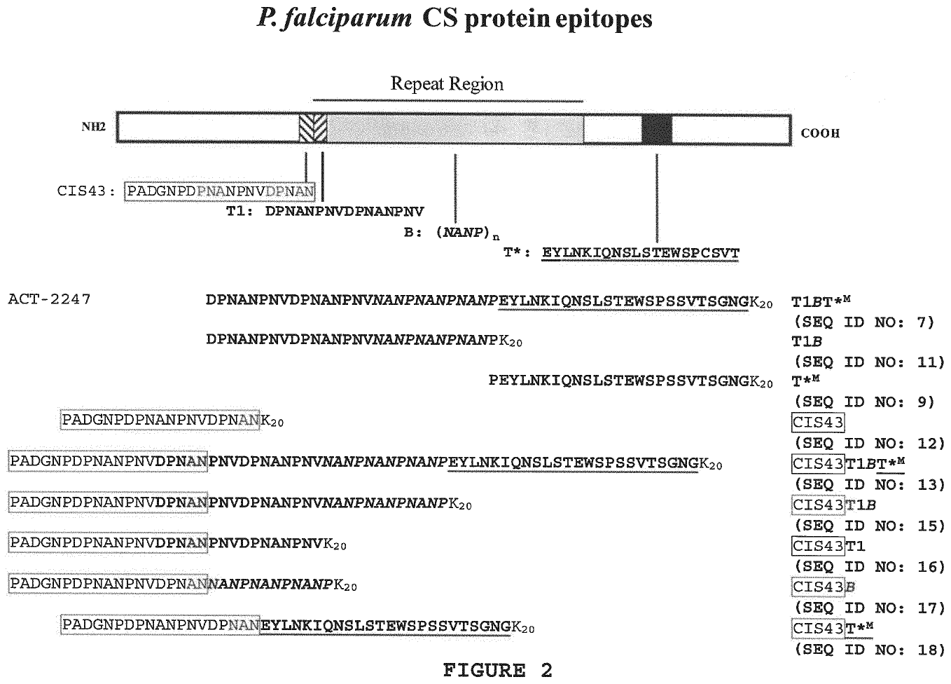

The above-described and other features will be appreciated and understood by those skilled in the art from the following detailed description, drawings, and appended claims.

### DETAILED DESCRIPTION

Disclosed herein are multilayer films comprising polypeptide epitopes from a Plasmodium protozoan, wherein the multilayer films are capable of eliciting an immune response in a host upon administration to the host. Previously, it was demonstrated that a particle comprising a multilayer film loaded with a peptide containing the T1BT* epitopes of the P. falciparum circumsporozoite [CS] protein elicits parasite-neutralizing antibody responses in mice, rabbits, and non-human primates, and also protects mice from challenge with a transgenic P. berghei expressing the central repeat region of P. falciparum CS. These films and particles are described in U.S. Pat. No. 9,433,671. Recently described is a CIS43 epitope identified as the binding target of human monoclonal antibody CIS43 that was isolated from a patient immunized with attenuated P. falciparum sporozoites. In passive transfer experiments, CIS43 protected mice from challenge with Plasmodium sporozoites expressing P. falciparum CS (either transgenic or endogenous). Binding studies showed that CIS43 interacted with CS in a two-step process, first binding with high affinity to the junction region epitope (101-NPDPNANPNVDPNAN-115; SEQ ID NO: 20) then with high affinity to residues in the B repeat epitope (133-NANPNANPNANPNAN-147: SEQ ID NO: 21) that overlaps the B repeat epitope 129-140 in T1BT*. It was hypothesized that this two-step binding process may induce conformational change in the CS protein and might protect it from proteolytic cleavage.

Described herein are multilayer films and particles containing the P. falciparum CIS43 epitope and one or more of the P. falciparum T1, B, and T* epitopes. In some embodiments, the T* epitope is a T*^{M} variant with a Cys to Ser substitution as described in U.S. Pat. No. 9,968,665. Specifically, in an embodiment, the films comprise one or more Plasmodium falciparum circumsporozoite protein antigens, wherein the circumsporozoite protein antigens include a CIS43 epitope and at least one of a T1 epitope, a B epitope, a T* epitope and a T*^{M} epitope. Also included are compositions comprising two or more different multilayer films.

As used herein, a P. falciparum circumsporozoite CIS43, T1, B, T* or T*^{M} epitope refers to a portion of the P. falciparum CS protein that is recognized by the immune system. Typically, epitopes have lengths of about 4 to about 25 amino acids in length, and do not include the entire P. falciparum CS protein.

It is expected that the addition of the CIS43 epitope to multilayer films containing at least one of a T1 epitope, a B epitope, a T* epitope and a T*^{M} epitope will result in a more potent malaria vaccine due to induction of higher titer and higher avidity antibody responses by the modified vaccine compared to current vaccines. The CS protein has SEQ ID NO: 1

As used herein, the Plasmodium falciparum circumsporozoite protein epitopes are:

T1:
(SEQ ID NO: 2)
DPNANPNVDPNANPNV
B:
(SEQ ID NO: 3)
NANP
T*:
(SEQ ID NO: 4)
EYLNKIQNSLSTEWSPCSVT
T*^{M} (Modified T*):
(SEQ ID NO: 5)
EYLNKIQNSLSTEWSPSSVT
CIS43:
(SEQ ID NO: 6)
PADGNPDPNANPNV

In certain embodiments, the T1, B, T*, or T*^{M} epitope, particularly the B epitope, is repeated 2 or more times.

Exemplary peptides include

T1BT*^{M}:
(SEQ ID NO: 7)
DPNANPNVDPNANPNVNANPNANPNANPEYLNKIQNSLSTEWSPSSVTS
GNGK₂₀
T1BT*:
(SEQ ID NO: 8)
DPNANPNVDPNANPNVNANPNANPNANPEYLNKIQNSLSTEWSPCSVTS
GNGK₂₀
T*^{M}:
(SEQ ID NO: 9)
DPNANPNVDPNANPNVNANPNANPNANPEYLNKIQNSLSTEWSPSSVTS
GNGK₂₀
T*:
(SEQ ID NO: 10)
DPNANPNVDPNANPNVNANPNANPNANPEYLNKIQNSLSTEWSPCSVTS
GNGK₂₀
T1B
(SEQ ID NO: 11)
DPNANPNVDPNANPNVNANPNANPNANP K₂₀

Exemplary inventive peptides include:

CIS43:
(SEQ ID NO: 12)
PADGNPDPNANPNVDPNANK₂₀
CIS43T1BT*^{M}:
(SEQ ID NO: 13)
PADGNPDPNANPNVDPNANPNVDPNANPNVNANPNANPNANP
EYLNKIQNSLSTEWSPSSVTSGNGK₂₀
CIS43T1BT*:
(SEQ ID NO: 14)
PADGNPDPNANPNVDPNANPNVDPNANPNVNANPNANPNANP
EYLNKIQNSLSTEWSPCSVTSGNGK₂₀
CIS43T1B:
(SEQ ID NO: 15)
PADGNPDPNANPNVDPNANPNVDPNANPNVNANPNANPNANPK₂₀
CIS43T1:
(SEQ ID NO: 16)
PADGNPDPNANPNVDPNANPNVDPNANPNVNANPNANPNANP K₂₀
CIS43B
(SEQ ID NO: 17)
PADGNPDPNANPNVDPNAN NANPNANPNANPK₂₀
CIS43T*^{M}:
(SEQ ID NO: 18)
PADGNPDPNANPNVDPNANEYLNKIQNSLSTEWSPSSVTSGNGK₂₀
CIS43T*:
(SEQ ID NO: 19)
PADGNPDPNANPNVDPNANEYLNKIQNSLSTEWSPCSVTSGNGK₂₀

Any combination of peptides may be employed. In an embodiment, a flexible linker such as SGS may be located between adjacent antigenic regions in a fusion polypeptide, such as CYS43-SGS-T1*^{M}.

In an embodiment, a multilayer film comprises a plurality of alternating oppositely charged polyelectrolyte layers, wherein one of the polyelectrolyte layers in the multilayer film comprises an antigenic polyelectrolyte, wherein the antigenic polyelectrolyte comprises a Plasmodium falciparum circumsporozoite CIS43 epitope and one or more of Plasmodium falciparum circumsporozoite T1, B, T* and T*^{M} epitopes covalently linked to the antigenic polyelectrolyte; wherein the polyelectrolytes in the multilayer film comprise a polycationic material or a polyanionic material having a molecular weight of greater than 1,000 and at least 5 charges per molecule. In this embodiment, the CIS43 epitope and one or more of T1, B, T* and T*^{M} epitopes are present on the same antigenic polyelectrolyte.

In another embodiment, a composition comprises a multilayer film comprising a plurality of alternating oppositely charged polyelectrolyte layers, wherein a first polyelectrolyte layer in the multilayer film comprises a first antigenic polyelectrolyte comprising a covalently linked Plasmodium falciparum circumsporozoite CIS43 epitope, and wherein a second polyelectrolyte layer in the multilayer film comprises a second antigenic polyelectrolyte comprising one or more of a Plasmodium falciparum circumsporozoite T1, B, T* and T*^{M} epitopes covalently linked to the second polyelectrolyte, wherein the first and second polyelectrolyte layers are the same or different layers, wherein the polyelectrolytes in the multilayer film comprise a polycationic material or a polyanionic material having a molecular weight of greater than 1,000 and at least 5 charges per molecule. In this embodiment, the CIS43 epitope and one or more of T1, B, T* and T*^{M} epitopes are on different antigenic polyelectrolytes, which polyelectrolytes can be in the same or in different layers of the multilayer film.

In a still further embodiment, a first multilayer film comprises a plurality of alternating oppositely charged polyelectrolyte layers, wherein a first polyelectrolyte layer in the first multilayer film comprises a first antigenic polyelectrolyte comprising a covalently linked Plasmodium falciparum circumsporozoite CIS43 epitope, and a second multilayer film comprising a plurality of oppositely charged polyelectrolyte layers, wherein a second polyelectrolyte layer in the multilayer film comprises a second antigenic polyelectrolyte comprising one or more of Plasmodium falciparum circumsporozoite T1, B, T* and T*^{M} epitopes covalently linked to the second polyelectrolyte, wherein the polyelectrolytes in the second multilayer film comprise a polycationic material or a polyanionic material having a molecular weight of greater than 1,000 and at least 5 charges per molecule. In this embodiment, the CIS43 epitope and one or more of T1, B, T* and T*^{M} epitopes are in different multilayer films, which are then mixed. For example, in this embodiment, the CIS43 epitope and one or more of T1, B, T* and T*^{M} epitopes can be layered onto separate microparticles which are then mixed to form the composition.

Specifically, the multilayer films comprise alternating layers of alternating oppositely charged polyelectrolytes. Optionally, one or more of the polyelectrolytes is a polypeptide. In certain embodiments, the multilayer films comprise multiple epitopes from a Plasmodium protozoan. For example, first and second Plasmodium protozoan polypeptide epitopes can be attached to the same or different polyelectrolytes, and/or can be present in the same or different multilayer film. In one embodiment, first and second Plasmodium protozoan polypeptide epitopes are covalently attached to the same polyelectrolyte and thus are in the same multilayer film. In another embodiment, first and second Plasmodium protozoan polypeptide epitopes are covalently attached to different polyelectrolytes, but are layered within the same multilayer film. In yet another embodiment, first and second Plasmodium protozoan polypeptide epitopes are covalently attached to different polyelectrolytes, but are layered in different multilayer films which are subsequently mixed prior to administration.

In an embodiment, the first antigenic polyelectrolyte comprises the CIS43 epitope and at least one of a Plasmodium falciparum circumsporozoite of T1, B, T* and T*^{M} epitopes. In one embodiment, the first antigenic polyelectrolyte comprises the CIS43 epitope and two of the Plasmodium falciparum circumsporozoite T1, B and T* epitopes, such as CIS43T1T*, CIS43T1T*^{M}, CIS43T1B, CIS43BT*, or CIS43BT*^{M} in any order. In yet another embodiment, the first antigenic polyelectrolyte comprises all three of the Plasmodium falciparum circumsporozoite T1, B and T* epitopes.

In one embodiment, the first polyelectrolyte is a polypeptide. The epitopes can be contiguous on the polypeptide chain, or spaced by a spacer region. Similarly, the epitopes can be at the N-terminus of the polypeptide, the C-terminus of the polypeptide, or anywhere in between. The epitopes can be in a contiguous part of the polypeptide, or any or all of the epitopes can be separated by a spacer region.

In an embodiment, the first antigenic polyelectrolyte comprises the CIS43 epitope and a second polyelectrolyte comprises at least one of a Plasmodium falciparum circumsporozoite T1, B, T* and T*^{M} epitopes. In one embodiment, the second antigenic polyelectrolyte comprises two of the Plasmodium falciparum circumsporozoite T1, B and T* epitopes, such as TIT*, T1T*^{M}, T1B, BT*, or BT*^{M} in any order.

In one embodiment, the first and second polyelectrolytes are polypeptides. The epitopes can be contiguous on the polypeptide chain, or spaced by a spacer region. Similarly, the epitopes can be at the N-terminus of the polypeptide, the C-terminus of the polypeptide, or anywhere in between. The epitopes can be in a contiguous part of the polypeptide, or any or all of the epitopes can be separated by a spacer region.

It is noted that when the first antigenic polyelectrolye is a polypeptide, the polypeptide contains sufficient charge for deposition into a polypeptide multilayer film. In one embodiment, the net charge per residue of the polypeptide is greater than or equal to 0.1, 0.2, 0.3, 0.4 or 0.5 at pH 7.0, as explained herein.

In an embodiment, an antigenic polypeptide comprises a surface adsorption region which provides charge so that the polypeptide can be stably deposited into a multilayer film. In an embodiment, the surface adsorption region comprises 5 or more negatively or positively charged amino acid residues.

In an embodiment, the multilayer films retain at least half of their polyelectrolytes when incubated in phosphate buffered saline at 37° C. for 24 hours.

In another embodiment, instead of the Plasmodium falciparum circumsporozoite CIS43, T1, B, T* and T*^{M} epitopes being on the same polyelectrolyte, two or three epitopes can be presented on separate polyelectrolytes, and layered into the same multilayer film. Alternative, the CIS43, T1, B, T* and T*^{M} epitopes present on separate polyelectrolytes can be present in different multilayer films.

In certain embodiments, the multilayer films further comprise a toll-like receptor ligand. As used herein, toll-like receptor ligands, or TLR ligands, are molecules that bind to TLRs and either activate or repress TLR receptors. Activation of TLR signaling through recognition of pathogen-associated molecular patterns (PAMPs) and mimics leads to the transcriptional activation of genes encoding pro-inflammatory cytokines, chemokines and co-stimulatory molecules, which can control the activation of the antigen-specific adaptive immune response. TLRs have been pursued as potential therapeutic targets for various inflammatory diseases and cancer. Following activation, TLRs induce the expression of a number of protein families, including inflammatory cytokines, type I interferons, and chemokines. TLR receptor ligands can function as adjuvants for the immune response.

Exemplary TLR ligands include a TLR1 ligand, a TLR2 ligand, a TLR3 ligand, a TLR4 ligand, a TLR5 ligand, a TLR6 ligand, a TLR 7 ligand, a TLR8 ligand, a TLR9 ligand and combinations thereof.

Exemplary TLR1 ligands include bacterial lipopeptide. Exemplary TLR2 ligands include lipopeptides such as Pam3Cys ([N-palmitoyl-S-[2,3-bis(palmitoyloxy)propyl]cysteine]) and Pam2Cys (Pam2Cys [S-[2,3-bis(palmitoyloxy)propyl]cysteine]). Exemplary TLR6 ligands are diacyl lipopeptides. TLR1 and TLR6 require heterodimerization with TLR2 to recognize ligands. TLR1/2 are activated by triacyl lipoprotein (or a lipopeptide, such as Pam3Cys), whereas TLR6/2 are activated by diacyl lipoproteins (e g., Pam2Cys), although there may be some cross-recognition.

An exemplary TLR3 ligand is Poly(I:C). Exemplary TLR4 ligands are lipopolysaccharide (LPS) and monophospholipid A (MPL). An exemplary TLR5 ligand is flagellin. An exemplary TLR7 ligand is imiquimod. An exemplary TLR8 ligand is single-stranded RNA. An exemplary TLR9 ligand is unmethylated CpG Oligodeoxynucleotide DNA.

In one embodiment, the first, second or third antigenic polyelectrolyte, e.g., an antigenic polypeptide, has a TLR ligand covalently attached thereto. For example, Pam3Cys can be covalently coupled to a polypeptide chain by standard polypeptide synthesis chemistry.

In another embodiment, a substrate such as a template core has deposited thereon a TLR ligand prior to deposition of polyelectrolyte layers. In another embodiment, a TLR ligand is co-deposited with one or more polyelectrolyte layers during assembly of the multilayer film.

In one embodiment, the multilayer film is deposited on a core particle, such as a CaCO₃ nanoparticle, a latex particle, or an iron particle. Particle sizes on the order of 5 nanometers (nm) to 15 micrometers (μm) in diameter are particularly useful, as are larger particles having diameters of 1 μm or more, such as 3 μm diameter particles. Particles made of other materials can also be used as cores provided that they are biocompatible, have controllable size distribution, and have sufficient surface charge (either positive or negative) to bind polyelectrolyte peptides. Examples include nanoparticles and microparticles made of materials such as polylactic acid (PLA), polylactic acid glycolic acid copolymer (PLGA), polyethylene glycol (PEG), chitosan, hyaluronic acid, gelatin, or combinations thereof. Core particles could also be made of materials that are believed to be inappropriate for human use provided that they can be dissolved and separated from the multilayer film following film fabrication. Examples of the template core substances include organic polymers such as latex or inorganic materials such as silica.

Polyelectrolyte multilayer films are thin films (e.g., a few nanometers to micrometers thick) composed of alternating layers of oppositely charged polyelectrolytes. Such films can be formed by layer-by-layer assembly on a suitable substrate. In electrostatic layer-by-layer self-assembly (“LBL”), the physical basis of association of polyelectrolytes is electrostatic attraction. Film buildup is possible because the sign of the surface charge density of the film reverses on deposition of successive layers. The generality and relative simplicity of the LBL film process permits the deposition of many different types of polyelectrolyte onto many different types of surface. Polypeptide multilayer films are a subset of polyelectrolyte multilayer films, comprising at least one layer comprising a charged polypeptide, herein referred to as a designed polypeptide. A key advantage of polypeptide multilayer films over films made from other polymers is their biocompatibility. LBL films can also be used for encapsulation. Applications of polypeptide films and microcapsules include, for example, nano-reactors, biosensors, artificial cells, and drug delivery vehicles.

The term “polyelectrolyte” includes polycationic and polyanionic materials having a molecular weight of greater than 1,000 and at least 5 charges per molecule. Suitable polycationic materials include, for example, polypeptides and polyamines. Polyamines include, for example, a polypeptide such as poly-L-lysine (PLL) or poly-L-ornithine, polyvinyl amine, poly(aminostyrene), poly(aminoacrylate), poly (N-methyl aminoacrylate), poly (N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), poly (diallyl dimethylammonium chloride), poly(N,N,N-trimethylaminoacrylate chloride), poly(methyacrylamidopropyltrimethyl ammonium chloride), chitosan and combinations comprising one or more of the foregoing polycationic materials. Suitable polyanionic materials include, for example, a polypeptide such as poly-L-glutamic acid (PGA) and poly-L-aspartic acid, a nucleic acid such as DNA and RNA, alginate, carrageenan, furcellaran, pectin, xanthan, hyaluronic acid, heparin, heparan sulfate, chondroitin sulfate, dermatan sulfate, dextran sulfate, poly(meth)acrylic acid, oxidized cellulose, carboxymethyl cellulose, acidic polysaccharides, and croscarmelose, synthetic polymers and copolymers containing pendant carboxyl groups, and combinations comprising one or more of the foregoing polyanionic materials. In one embodiment, the Plasmodium protozoan epitope and the polyelectrolyte have the same sign of charge.

In one embodiment, one or more polyelectrolyte layers of the film, optionally including the polyelectrolyte comprising the Plasmodium protozoan epitope, is a designed polypeptide. In one embodiment, the design principles for polypeptides suitable for electrostatic layer-by-layer deposition are elucidated in U.S. Patent Publication No. 2005/0069950, incorporated herein by reference for its teaching of polypeptide multilayer films. Briefly, the primary design concerns are the length and charge of the polypeptide. Electrostatics is the most important design concern because it is the basis of LBL. Without suitable charge properties, a polypeptide may not be substantially soluble in aqueous solution at pH 4 to 10 and cannot readily be used for the fabrication of a multilayer film by LBL. Other design concerns include the physical structure of the polypeptides, the physical stability of the films formed from the polypeptides, and the biocompatibility and bioactivity of the films and the constituent polypeptides.

A designed polypeptide means a polypeptide that has sufficient charge for stable binding to an oppositely charged surface, that is, a polypeptide that can be deposited into a layer of a multilayer film wherein the driving force for film formation is electrostatics. A short stable film is a film that once formed, retains more than half its components after incubation in PBS at 37° C. for 24 hours. In specific embodiments, a designed polypeptide is at least 15 amino acids in length and the magnitude of the net charge per residue of the polypeptide is greater than or equal to 0.1, 0.2, 0.3, 0.4 or 0.5 at pH 7.0. Positively-charged (basic) naturally-occurring amino acids at pH 7.0 are arginine (Arg), histidine (His), ornithine (Orn), and lysine (Lys). Negatively-charged (acidic) naturally-occurring amino acid residues at pH 7.0 are glutamic acid (Glu) and aspartic acid (Asp). A mixture of amino acid residues of opposite charge can be employed so long as the overall net ratio of charge meets the specified criteria. In one embodiment, a designed polypeptide is not a homopolymer. In another embodiment, a designed polypeptide is unbranched.

One design concern is control of the stability of polypeptide LBL films. Ionic bonds, hydrogen bonds, van der Waals interactions, and hydrophobic interactions contribute to the stability of multilayer films. In addition, covalent disulfide bonds formed between sulfhydryl-containing amino acids in the polypeptides within the same layer or in adjacent layers can increase structural strength. Sulfhydryl-containing amino acids include cysteine and homocysteine and these residues can be readily incorporated into synthetic designed peptides. In addition sulfhydryl groups can be incorporated into polyelectrolyte homopolymers such as poly-L-lysine or poly-L-glutamic acid by methods well described in the literature. Sulfhydryl-containing amino acids can be used to “lock” (bond together) and “unlock” layers of a multilayer polypeptide film by a change in oxidation potential. Also, the incorporation of a sulfhydryl-containing amino acid in a designed polypeptide enables the use of relatively short peptides in thin film fabrication, by virtue of intermolecular disulfide bond formation.

In one embodiment, the designed sulfhydryl-containing polypeptides, whether synthesized chemically or produced in a host organism, are assembled by LBL in the presence of a reducing agent to prevent premature disulfide bond formation. Following film assembly, the reducing agent is removed and an oxidizing agent is added. In the presence of the oxidizing agent disulfide bonds form between sulfhydryl groups, thereby “locking” together the polypeptides within layers and between layers where thiol groups are present. Suitable reducing agents include dithiothreitol (DTT), 2-mercaptoethanol (BME), reduced glutathione, tris(2-carboxyethyl)phosphine hydrochloride (TCEP), and combinations of more than one of these chemicals. Suitable oxidizing agents include oxidized glutathione, tert-butylhydroperoxide (t-BHP), thimerosal, diamide, 5,5′-dithio-bis-(2-nitro-benzoic acid) (DTNB), 4,4′-dithiodipyridine, sodium bromate, hydrogen peroxide, sodium tetrathionate, porphyrindin, sodium orthoiodosobenzoate, and combinations of more than one of these chemicals.

As an alternative to disulfide bonds, chemistries that produce other covalent bonds can be used to stabilize LBL films. For films comprised of polypeptides, chemistries that produce amide bonds are particularly useful. In the presence of appropriate coupling reagents, acidic amino acids (those with side chains containing carboxylic acid groups such as aspartic acid and glutamic acid) will react with amino acids whose side chains contain amine groups (such as lysine and ornithine) to form amide bonds. Amide bonds are more stable than disulfide bonds under biological conditions and amide bonds will not undergo exchange reactions. Many reagents can be used to activate polypeptide side chains for amide bonding. Carbodiimide reagents, such as the water soluble 1-ethyl-3-(3-dimethylaminopropyl) carbodiimide (EDC) will react with aspartic acid or glutamic acid at slightly acidic pH, forming an intermediate product that will react irreversibly with an amine to produce an amide bond. Additives such as N-hydroxysuccinimide are often added to the reaction to accelerate the rate and efficiency of amide formation. After the reaction the soluble reagents are removed from the nanoparticles or microparticles by centrifugation and aspiration. Examples of other coupling reagents include diisopropylcarbodiimide, HBTU, HATU, HCTU, TBTU, and PyBOP. Examples of other additives include sulfo-N-hydroxysuccinimide, 1-hydroxbenzotriazole, and 1-hydroxy-7-aza-benzotriazole. The extent of amide cross linking can be controlled by modulating the stoichiometry of the coupling reagents, the time of reaction, or the temperature of the reaction, and can be monitored by techniques such as Fourier transform—infrared spectroscopy (FT-IR).

Covalently cross-linked LBL films have desirable properties such as increased stability. Greater stability allows for more stringent conditions to be used during nanoparticle, microparticle, nanocapsule, or microcapsule fabrication. Examples of stringent conditions include high temperatures, low temperatures, cryogenic temperatures, high centrifugation speeds, high salt buffers, high pH buffers, low pH buffers, filtration, and long term storage.

A method of making a polyelectrolyte multilayer film comprises depositing a plurality of layers of oppositely charged chemical species on a substrate. In one embodiment, at least one layer comprises a designed polypeptide. Successively deposited polyelectrolytes will have opposite net charges. In one embodiment, deposition of a polyelectrolyte comprises exposing the substrate to an aqueous solution comprising a polyelectrolyte at a pH at which it has a suitable net charge for LBL. In other embodiments, the deposition of a polyelectrolyte on the substrate is achieved by sequential spraying of solutions of oppositely charged polypeptides. In yet other embodiments, deposition on the substrate is by simultaneous spraying of solutions of oppositely charged polyelectrolytes.

In the LBL method of forming a multilayer film, the opposing charges of the adjacent layers provide the driving force for assembly. It is not critical that polyelectrolytes in opposing layers have the same net linear charge density, only that opposing layers have opposite charges. One standard film assembly procedure by deposition includes forming aqueous solutions of the polyions at a pH at which they are ionized (i.e., pH 4-10), providing a substrate bearing a surface charge, and alternating immersion of the substrate into the charged polyelectrolyte solutions. The substrate is optionally washed in between deposition of alternating layers.

The concentration of polyelectrolyte suitable for deposition of the polyelectrolyte can readily be determined by one of ordinary skill in the art. An exemplary concentration is 0.1 to 10 mg/mL. For typical non-polypeptide polyelectrolytes such as poly(acrylic acid) and poly(allylamine hydrochloride), typical layer thicknesses are about 3 to about 5 Å, depending on the ionic strength of solution. Short polyelectrolytes typically form thinner layers than long polyelectrolytes. Regarding film thickness, polyelectrolyte film thickness depends on humidity as well as the number of layers and composition of the film. For example, PLL/PGA films 50 nm thick shrink to 1.6 nm upon drying with nitrogen. In general, films of 1 nm to 100 nm or more in thickness can be formed depending on the hydration state of the film and the molecular weight of the polyelectrolytes employed in the assembly.

In addition, the number of layers required to form a stable polyelectrolyte multilayer film will depend on the polyelectrolytes in the film. For films comprising only low molecular weight polypeptide layers, a film will typically have 4 or more bilayers of oppositely charged polypeptides. For films comprising high molecular weight polyelectrolytes such as poly(acrylic acid) and poly(allylamine hydrochloride), films comprising a single bilayer of oppositely charged polyelectrolyte can be stable. Studies have shown that polyelectrolyte films are dynamic. The polyelectrolytes contained within a film can migrate between layers and can exchange with soluble polyelectrolytes of like charge when suspended in a polyelectrolyte solution. Moreover polyelectrolyte films can disassemble or dissolve in response to a change in environment such as temperature, pH, ionic strength, or oxidation potential of the suspension buffer. Thus some polyelectrolytes and particularly peptide polyelectrolytes exhibit transient stability. The stability of peptide polyelectrolyte films can be monitored by suspending the films in a suitable buffer under controlled conditions for a fixed period of time, and then measuring the amounts of the peptides within the film with a suitable assay such as amino acid analysis, HPLC assay, or fluorescence assay. Peptide polyelectrolyte films are most stable under conditions that are relevant to their storage and usage as vaccines, for example in neutral buffers and at ambient temperatures such as 4° C. to 37° C. Under these conditions stable peptide polyelectrolyte films will retain most of their component peptides for at least 24 hours and often up to 14 days and beyond.

In one embodiment, a designed polypeptide comprises one or more surface adsorption regions covalently linked to one or more Plasmodium protozoan epitopes, wherein the designed polypeptide and the one or more surface adsorption regions have the same sign of charge, that is, are both positively or both negatively charged overall. As used herein, a surface adsorption region is a charged region of a designed polypeptide that advantageously provides sufficient charge so that a peptide containing an epitope from a Plasmodium protozoan, for example, can be deposited into a multilayer film. In one embodiment, the one or more surface adsorption regions and the one or more Plasmodium protozoan epitopes have the same net polarity. In another embodiment, the solubility of the designed polypeptide at pH 4 to 10 is greater than or equal to about 0.1 mg/mL. In another embodiment, the solubility of the designed polypeptide at pH 4 to 10 is greater than or equal to about 1 mg/mL. The solubility is a practical limitation to facilitate deposition of the polypeptides from aqueous solution. A practical upper limit on the degree of polymerization of an antigenic polypeptide is about 1,000 residues. It is conceivable, however, that longer composite polypeptides could be realized by an appropriate method of synthesis.

In one embodiment, a designed polypeptide comprises a single antigenic Plasmodium protozoan epitope flanked by two surface adsorption regions, an N-terminal surface adsorption region and a C-terminal surface adsorption region. In another embodiment, a designed polypeptide comprises a single antigenic Plasmodium protozoan epitope flanked by one surface adsorption region linked to the N-terminus of the Plasmodium protozoan epitope. In another embodiment, a designed polypeptide comprises a single antigenic Plasmodium protozoan epitope flanked by one surface adsorption regions linked to the C-terminus of the Plasmodium protozoan epitope.

Each of the independent regions (e.g., Plasmodium protozoan epitopes and surface adsorption regions) of the designed polypeptide can be synthesized separately by solution phase peptide synthesis, solid phase peptide synthesis, or genetic engineering of a suitable host organism. Solution phase peptide synthesis is the method used for production of most of the approved peptide pharmaceuticals on the market today. A combination of solution phase and solid phase methods can be used to synthesize relatively long peptides and even small proteins. Peptide synthesis companies have the expertise and experience to synthesize difficult peptides on a fee-for-service basis. The syntheses are performed under good manufacturing practices (GMP) conditions and at a scale suitable for clinical trials and commercial drug launch.

Alternatively, the various independent regions can be synthesized together as a single polypeptide chain by solution-phase peptide synthesis, solid phase peptide synthesis or genetic engineering of a suitable host organism. The choice of approach in any particular case will be a matter of convenience or economics.

If the various Plasmodium protozoan epitopes and surface adsorption regions are synthesized separately, once purified, for example, by ion exchange chromatography or by high performance liquid chromatography, they are joined by peptide bond synthesis. That is, the N-terminus of the surface adsorption region and the C-terminus of the Plasmodium protozoan epitope are covalently joined to produce the designed polypeptide. Alternatively, the C-terminus of the surface adsorption region and the N-terminus of the Plasmodium protozoan epitope are covalently joined to produce the designed polypeptide. The individual fragments can be synthesized by solid phase methods and obtained as fully protected, fully unprotected, or partially protected segments. The segments can be covalently joined in a solution phase reaction or solid phase reaction. If one polypeptide fragment contains a cysteine as its N-terminal residue and the other polypeptide fragment contains a thioester or a thioester precursor at its C-terminal residue the two fragments will couple spontaneously in solution by a specific reaction commonly known (to those skilled in the art) as Native Ligation. Native Ligation is a particularly attractive option for designed peptide synthesis because it can be performed with fully deprotected or partially protected peptide fragments in aqueous solution and at dilute concentrations.

In one embodiment, the Plasmodium protozoan epitopes and/or surface adsorption regions are joined by peptidic or non-peptidic linkages as described in U.S. Pat. No. 7,723,294, incorporated herein by reference for its teaching of the use of non-peptidic linkages to join segments of polypeptides for use in multilayer films. Suitable non-peptidic linkers include, for example, alkyl linkers such as —NH—(CH₂), —C(O)—, wherein s=2-20. Alkyl linkers are optionally substituted by a non-sterically hindering group such as lower alkyl (e.g., C₁-C₆), lower acyl, halogen (e.g., Cl, Br), CN, NH₂, phenyl, and the like. Another exemplary non-peptidic linker is a polyethylene glycol linker such as —NH—(CH₂—CH₂—O)ₙ, —C(O)— wherein n is such that the linker has a molecular weight of 100 to 5000 Da, specifically 100 to 500 Da. Many of the linkers described herein are available from commercial vendors in a form suitable for use in solid phase peptide synthesis.

In one embodiment, one or more of the polypeptide epitopes from a Plasmodium protozoan is covalently attached to one or more of the polyelectrolyes, such as a polypeptide or other polyelectrolyte, through covalent bonds. Examples of suitable covalent bonds include amides, esters, ethers, thioethers, and disulfides. One skilled in the art can take advantage of a range of functional groups found within the epitope peptide to engineer a bond to a suitable electrolyte. For instance, a carboxylic acid in the epitope peptide can be found either at the C-terminal or on the side chain of amino acids aspartic acid or glutamic acid. Carboxylic acids can be activated with suitable peptide coupling reagents such as 1-ethyl-3-(3-dimethylaminopropyl) carbodiimide (EDC) for reaction with primary or secondary amines that are found in peptide polyelectrolytes such as poly-L-lysine. The resulting amide bond is stable under ambient conditions. Conversely, the acid groups in a peptide polyelectrolyte can be activated with EDC for reaction with amine groups in the epitope peptide. Useful amine groups can be found at the epitope peptide's N-terminal or on the side chain of lysine residues.

Epitope peptides can also be attached to polyelectrolytes via disulfide bonds. Polyelectrolytes such as PGA or PLL can be chemically modified so that a fraction of their side chains contain sulfhydryl groups. In the presence of a suitable oxidant, those sulfydryls will react with the sulfhydryl group of a cysteine residue contained within the epitope peptide. The cysteine can either be a native cysteine from the protein sequence of a pathogen such as a Plasmodium protozoan or it can be a non-native cysteine that was intentionally incorporated into the epitope during peptide synthesis. Suitable oxidants include DTNB, 2,2′-dithiopyridine, hydrogen peroxide, cystine, and oxidized glutathione. The attachment of epitope peptides to polyelectrolytes via disulfide bonds is particularly useful. The disulfides are stable under normal conditions of film fabrication and storage but are readily cleaved by reducing agents found naturally in cells, which frees up the epitope peptide for immune processing.

Epitope peptides can also be attached to polyelectrolytes via thioether bonds. Synthetic epitope peptides can be synthesized with appropriate electrophiles such as haloacetyl groups which react specifically with sulfhydryls. For instance, an epitope peptide containing a chloroacetyl at its N-terminal will form a stable bond to sulfhydryl bearing polyelectrolytes such as PGA-SH described above.

Epitope peptides can also be attached covalently to polyelectrolytes through bifunctional linker molecules. Bifunctional linkers usually contain two electrophilic groups that can react with nucleophiles present on either the epitope peptide or the polyelectrolyte molecule. Two classes of linker molecules are sold commercially, homobifunctional linkers and heterobifunctional linkers. Homobifunctional linkers contain two copies of an electrophilic group joined by a nonreactive spacer. Often the electophiles are active esters, such as N-hydroxysuccinimide (NHS) esters or sulfo-N-hyrdoxysuccinimide esters (sulfo NHS) which react with nucleophilic amines. Examples of homobifunctional NHS esters include bis(sulfosuccinimidyl) suberate, disuccinimidyl glutarate, dithiobis(succinimidyl) propionate, disuccinimidyl suberate, disuccinimidyl tartrate. Sometimes the electophiles are aldehyde groups that form imides with nucleophilic amines on the epitope and polyelectrolyte molecules. The imide bonds are transiently stable but can be converted to stable structures with reducing agents such as sodium borohydride or catalytic hydrogenation. The most commonly used homobifunctional aldehyde linker is glutaraldehyde.

Other commonly used homobifunctional linkers contain electrophiles that react specifically with nucleophilic thiols, which can be used to link cysteine containing epitope peptides to sulfhydryl containing polyelectrolytes as described above. Examples of sulfhydryl specific homobifunctional linkers include 1,4-bismaleimidobutane, 1,4 bismaleimidyl-2,3-dihydroxybutane, vbismaleimidohexane, bis-maleimidoethane, 1,4-di-[3′-(2′-pyridyldithio)-propionamido]butane, dithio-bismaleimidoethane, 1,6-hexane-bis-vinylsulfone.

Members of the heterobifunctional class of cross linking reagents contain two different reactivity groups, often but not always electrophiles, which react specifically with different functional groups in substrate molecules. Particularly useful are linkers that contain one electrophilic group that is specific for a sulfhydryl and another electrophile that is specific for an amine. Examples of these reagents include N-sulfosuccinimidyl[4-iodoacetyl]aminobenzoate, N-succinimidyl[4-iodoacetyl]aminobenzoate, succinimidyl 3-[bromoacetamido]propionate, N-succinimidyl iodoacetate, sulfosuccinimidyl 4[N-maleimidomethyl]cyclohexane-1-carboxylate, succinimidyl 4-[N-maleimidomethyl]cyclohexane-1-carboxylate, ([N-e-maleimidocaproyloxy]sulfosuccinimide ester, m-maleimidobenzoyl-N-hydroxysulfosuccinimide ester, N-succinimidyl 3-(2-pyridyldithio)-propionate, succinimidyl 6-(3-[2-pyridyldithio]-propionamido)hexanoate, 4-succinimidyloxycarbonyl-methyl-a-[2-pyridyldithio]toluene.

The wide range of functionality that is normally present in both epitope peptides and polyelectrolytes or which can easily be installed in either molecule allows one to choose a linking strategy that best fits the substrates of interest. A likely example is the linking of a cysteine containing epitope peptide to PLL.

The polypeptide segments can be joined in a variety of ways, depending upon the chemistry of the non-peptidic linker. For example, the N-terminus of the first polypeptide segment is joined to the C-terminus of the second polypeptide segment; the N-terminus of the first polypeptide segment is joined to the N-terminus of the second polypeptide segment; the C-terminus of the first polypeptide segment is joined to the C-terminus of the second polypeptide segment; the C-terminus of the first polypeptide segment is joined to the N-terminus of the second polypeptide segment; the C-terminus or the N-terminus of the first polypeptide segment is joined to a pendant side chain of the second polypeptide segment; or the C-terminus or the N-terminus of the second polypeptide segment is joined to a pendant side chain of the first polypeptide segment. Regardless of the point of attachment, however, the first and second segments are covalently joined by a non-peptidic linker.

In one embodiment, a designed polypeptide is a unique combination of covalently attached one or more surface adsorption region(s) and one or more Plasmodium protozoan epitope(s). There is no particular limitation on the length of the Plasmodium protozoan epitopes, which can be linear epitopes or conformational epitopes. Epitopes can comprise anywhere from about three amino acid resides up to several hundred amino acid residues for complex conformational epitopes.

In one embodiment, a designed polypeptide comprises one Plasmodium protozoan epitope and one surface adsorption region. In another embodiment, a designed polypeptide comprises one Plasmodium protozoan epitope and two surface adsorption regions, one attached to the N-terminus of the Plasmodium protozoan epitope and one attached to the C-terminus of the Plasmodium protozoan epitope. The purpose of the surface adsorption region(s) is to enable adsorption of the polypeptide onto an oppositely charged surface in order to build a multilayer film.

The number of surface adsorption regions in a designed polypeptide relative to the number and/or length of the Plasmodium protozoan epitopes is related to the solubility requirement. For example, if the Plasmodium protozoan epitope is a short amino acid sequence of, for example, three amino acid residues, only one surface adsorption region of at least eight amino acid residues will be required to adsorb the designed polypeptide onto a suitably charged surface. If, by contrast, the Plasmodium protozoan epitope is a soluble folded structural domain of a protein comprising, for example, 120 amino acid residues, two surface adsorption regions may be required to impart enough charge for the designed polypeptide to be water soluble and suitable for adsorption. The surface adsorption regions could be contiguous and located at the N-terminus of the domain, contiguous and located at the C-terminus of the domain, or noncontiguous with one at the N-terminus and one at the C-terminus. Additionally, a Plasmodium protozoan epitope may contain a charged segment (either negatively charged or positively charged) within its native sequence that can serve as a surface adsorption region.

A polypeptide or antigen may contain one or more distinct antigenic determinants. An antigenic determinant may refer to an immunogenic portion of a multichain protein.

Methods and techniques for determining the location and composition of an antigenic determinant or epitope for a specific antibody are well known in the art. These techniques can be used to identify and/or characterize epitopes for use as Plasmodium protozoan epitopes. In one embodiment, mapping/characterization methods of an epitope for an antigen specific antibody can be determined by epitope “foot-printing” using chemical modification of the exposed amines/carboxyls in the antigenic protein. One example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuteriated. Relevant regions may be identified at this point by peptic proteolysis, fast microbore high-performance liquid chromatography separation, and/or electrospray ionization mass spectrometry.

In another embodiment, a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with ¹⁵N so that only signals corresponding to the antigen and no signals from the antigen binding peptide are seen in the NMR spectrum. Antigen signals originating from amino acids involved in the interaction with the antigen binding peptide typically will shift position in the spectra of the complex compared to the spectra of the free antigen, and the amino acids involved in the binding may be identified that way.

In another embodiment, epitope mapping/characterization may be done by peptide scanning. In this approach, a series of overlapping peptides spanning the full length of the polypeptide chain of an antigen are prepared and tested individually with regard to immunogenicity. The antibody titer of the corresponding peptide antigen is determined by a standard method, e.g., enzyme-linked immunosorbent assay. The various peptides can then be ranked with regard to immunogenicity, providing an empirical basis for selection of peptide design for vaccine development.

In another embodiment, protease digestion techniques may also be useful in the context of epitope mapping and identification. Antigenic determinant-relevant regions/sequences may be determined by protease digestion, e.g. by using trypsin in a ratio of about 1:50 to antigenic protein overnight (0/N) digestion at 37° C. and pH 7-8, followed by mass spectrometry (MS) analysis for peptide identification. The peptides protected from trypsin cleavage by the antigenic protein may subsequently be identified by comparison of samples subjected to trypsin digestion and samples incubated with CD38BP and then subjected to digestion by e.g. trypsin (thereby revealing a foot print for the binder). Other enzymes like chymotrypsin, pepsin, etc., may also or alternatively be used in a similar epitope characterization method. Moreover, protease digestion can provide a quick method for determining the location of a potential antigenic determinant sequence within a known antigenic protein using a known antibody. In another embodiment, protease digestion techniques may also be useful in the context of epitope mapping and identification.

Further disclosed herein is an immunogenic composition, said immunogenic composition comprising a multilayer film comprising two or more layers of polyelectrolytes, wherein adjacent layers comprise oppositely charged polyelectrolytes, wherein one layer comprises a Plasmodium protozoan epitope. The immunogenic composition optionally further comprises one or more layers comprising a designed polypeptide.

In one embodiment, an immunogenic composition comprises a plurality of Plasmodium protozoan epitopes, either on the same or different polyelectrolytes, for example, designed polypeptides. The plurality of antigenic determinants may be from the same or different infectious agents. In one embodiment, the immunogenic composition comprises a plurality of unique antigenic polyelectrolytes. In another embodiment, the immunogenic composition comprises a plurality of immunogenic polyelectrolytes comprising multiple Plasmodium protozoan epitopes within each polyelectrolyte. An advantage of these immunogenic compositions is that multiple antigenic determinants or multiple conformations of a single linear antigenic determinant can be present in a single synthetic vaccine particle. Such compositions with multiple antigenic determinants can potentially yield antibodies against multiple epitopes, increasing the odds that at least some of the antibodies generated by the immune system of the organism will neutralize the pathogen or target specific antigens on cancer cells, for example.

The immunogenicity of an immunogenic composition may be enhanced in a number of ways. In one embodiment, the multilayer film optionally comprises one or more additional immunogenic bioactive molecules. Although not necessary, the one or more additional immunogenic bioactive molecules will typically comprise one or more additional antigenic determinants. Suitable additional immunogenic bioactive molecules include, for example, a drug, a protein, an oligonucleotide, a nucleic acid, a lipid, a phospholipid, a carbohydrate, a polysaccharide, a lipopolysaccharide, a low molecular weight immune stimulatory molecule, or a combination comprising one or more of the foregoing bioactive molecules. Other types of additional immune enhancers include a functional membrane fragment, a membrane structure, a virus, a pathogen, a cell, an aggregate of cells, an organelle, or a combination comprising one or more of the foregoing bioactive structures.

In one embodiment, the multilayer film optionally comprises one or more additional bioactive molecules. The one or more additional bioactive molecule can be a drug. Alternatively, the immunogenic composition is in the form of a hollow shell or a coating surrounding a core. The core comprises a variety of different encapsulants, for example, one or more additional bioactive molecules, including, for example, a drug. Thus, the immunogenic compositions designed as described herein could also be used for combined therapy, e.g., eliciting an immune response and for targeted drug delivery. Micron-sized “cores” of a suitable therapeutic material in “crystalline” form can be encapsulated by immunogenic composition comprising the antigenic polypeptides, and the resulting microcapsules could be used for drug delivery. The core may be insoluble under some conditions, for instance high pH or low temperature, and soluble under the conditions where controlled release will occur. The surface charge on the crystals can be determined by ζ-potential measurements (used to determine the charge in electrostatic units on colloidal particles in a liquid medium). The rate at which microcapsule contents are released from the interior of the microcapsule to the surrounding environment will depend on a number of factors, including the thickness of the encapsulating shell, the antigenic polypeptides used in the shell, the presence of disulfide bonds, the extent of cross-linking of peptides, temperature, ionic strength, and the method used to assemble the peptides. Generally, the thicker the capsule, the longer the release time.

In another embodiment, the additional immunogenic biomolecule is a nucleic acid sequence capable of directing host organism synthesis of a desired immunogen or interfering with the expression of genetic information from a pathogen. In the former case, such a nucleic acid sequence is, for example, inserted into a suitable expression vector by methods known to those skilled in the art. Expression vectors suitable for producing high efficiency gene transfer in vivo include retroviral, adenoviral and vaccinia viral vectors. Operational elements of such expression vectors include at least one promoter, at least one operator, at least one leader sequence, at least one terminator codon, and any other DNA sequences necessary or preferred for appropriate transcription and subsequent translation of the vector nucleic acid. In particular, it is contemplated that such vectors will contain at least one origin of replication recognized by the host organism along with at least one selectable marker and at least one promoter sequence capable of initiating transcription of the nucleic acid sequence. In the latter case, multiple copies of such a nucleic acid sequence will be prepared for delivery, for example, by encapsulation of the nucleic acids within a polypeptide multilayer film in the form of a capsule for intravenous delivery.

In construction of a recombinant expression vector, it should additionally be noted that multiple copies of the nucleic acid sequence of interest and its attendant operational elements may be inserted into each vector. In such an embodiment, the host organism would produce greater amounts per vector of the desired protein. The number of multiple copies of the nucleic acid sequence which may be inserted into the vector is limited only by the ability of the resultant vector due to its size, to be transferred into and replicated and transcribed in an appropriate host microorganism.

In a further embodiment, the immunogenic composition comprises a mixture of antigenic polyelectrolytes/immunogenic bioactive molecules. These may be derived from the same antigen, they may be different antigens from the same infectious agent or disease, or they may be from different infectious agents or diseases. The complex or mixture will therefore raise an immune response against a number of antigens and possibly a number of infectious agents or diseases as specified by the antigenic peptide/protein components of the delivery system.

In one embodiment, the multilayer film/immunogenic composition evokes a response from the immune system to a pathogen. In one embodiment, a vaccine composition comprises an immunogenic composition in combination with a pharmaceutically acceptable carrier. Thus a method of vaccination against a pathogenic disease comprises the administering to a subject in need of vaccination an effective amount of the immunogenic composition.

Pharmaceutically acceptable carriers include, but are not limited to, large, slowly metabolized macromolecules such as proteins, polysaccharides, polylactic acids, polyglycolic acids, polymeric amino acids, amino acid copolymers, inactive virus particles, and the like. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition can also contain liquids, such as water, saline, glycerol, and ethanol, as well as substances such as wetting agents, emulsifying agents, or pH buffering agents. Liposomes can also be used as carriers.

A method of eliciting an immune response against a disease or pathogen in a vertebrate (e.g., vaccination) comprises administering an immunogenic composition comprising a multilayer film comprising a Plasmodium protozoan epitope. In one embodiment, the polyelectrolyte containing the Plasmodium protozoan epitope is in the most exterior or solvent-exposed layer of the multilayer film. The immunogenic composition can be administered orally, intranasally, intravenously, intramuscularly, subcutaneously, intraperitoneally, sublingually, intradermally, pulmonary, or transdermally, either with or without a booster dose. Generally, the compositions are administered in a manner compatible with the dosage formulation, and in such amount as will be prophylactically and/or therapeutically effective. Precise amounts of immunogenic composition to be administered depend on the judgment of the practitioner and may be peculiar to each subject. It will be apparent to those of skill in the art that the therapeutically effective amount of an immunogenic composition will depend, inter alia, upon the administration schedule, the unit dose of antigen administered, whether the compositions are administered in combination with other therapeutic agents, and the immune status and health of the recipient. A therapeutically effective dosage can be determined by the ordinary skilled medical worker based on patient characteristics (age, weight, sex, condition, complications, other diseases, etc.), as is well known in the art. Furthermore, as further routine studies are conducted, more specific information will emerge regarding appropriate dosage levels for treatment of various conditions in various patients, and the ordinary skilled worker, considering the therapeutic context, age and general health of the recipient, is able to ascertain proper dosing.

The immunogenic composition optionally comprises an adjuvant. Adjuvants in general comprise substances that boost the immune response of the host in a non-specific manner. Selection of an adjuvant depends on the subject to be vaccinated. Preferably, a pharmaceutically acceptable adjuvant is used. For example, a vaccine for a human should avoid oil or hydrocarbon emulsion adjuvants, including complete and incomplete Freund's adjuvant. One example of an adjuvant suitable for use with humans is alum (alumina gel). A vaccine for an animal, however, may contain adjuvants not appropriate for use with humans.

It is contemplated that an immune response may be elicited via presentation of any protein or peptide capable of eliciting such a response. In one embodiment, the antigen is a key epitope, which gives rise to a strong immune response to a particular agent of infectious disease, i.e., an immunodominant epitope. If desired, more than one antigen or epitope may be included in the immunogenic composition in order to increase the likelihood of an immune response.

In one embodiment, multiple Plasmodium protozoan peptide or protein epitopes are incorporated into an LBL film. The distinct epitopes can by synthesized or expressed within a single designed peptide molecule. Placing multiple epitopes within a single designed peptide is expected to have certain advantages. For example it should simplify the LBL fabrication process and increase reproducibility. Additionally, placing multiple epitopes within a single designed peptide will lock the molar ratios of the distinct epitopes in a desired ratio, for example 1:1.

Alternatively the epitopes can be incorporated into separate designed peptides. The designed peptides are incorporated into an LBL film during one or more layering steps. Fabrication of films using multiple distinct designed peptides can also present certain advantages. It should simplify designed peptide synthesis reducing costs. It will also enable the relative doses of each designed peptide within the film to be varied and optimized. If, for example, preclinical or clinical biological data indicated that an optimal vaccine should contain five copies of one epitope to every copy of a second epitope (5:1 ratio) the separate epitope designed peptide approach would facilitate the manufacture of such a vaccine.

Designed peptides adsorb to the surface of an LBL films by virtue of the electrostatic attraction between the charged surface adsorption regions(s) of the designed peptide and the oppositely charged surface of the film. The efficiency of adsorption will depend largely upon the composition of the surface adsorption region(s). Thus designed peptides with different epitopes but similar surface adsorption regions(s) will adsorb with similar efficiency. To fabricate a film with two distinct designed peptides each at a 1:1 molar ratio one could mix the peptides at that molar ratio and deposit them simultaneously at a particular layer. Alternatively, one could deposit each peptide individually at separate layers. The molar ratio of peptides adsorbed will largely mirror that relative concentrations at which they were layered or the number of layering steps during which they were incorporated.

The quantity of designed peptides incorporated into an LBL film can be measured in a variety of ways. Quantitative amino acid analysis (AAA) is particularly well suited to this purpose. Films containing designed peptides are decomposed to their constituent amino acids by treatment with concentrated hydrochloric acid (6 M) and heating, typically at 115° C. for 15 hours. The amounts of each amino acid are then measured using chromatographic techniques well known to those skilled in the art. Amino acids that occur in only one of the designed peptides in a film can be used as tracers for that peptide. When designed peptides lack unique amino acids, non-natural amino acids (e.g. aminobutyric acid or homovaline) can be incorporated into designed peptides during synthesis. These tracer amino acids are readily identified during the AAA experiment and can be used to quantitate the amount of peptide in the film.

As used herein, a specific T-cell response is a response that is specific to an epitope of interest, specifically a Plasmodium protozoan epitope. A specific T-cell response is an IFNγ and/or an IL-5 T-cell response.

As used herein, a specific antibody response is a response that is specific to an epitope of interest, specifically a Plasmodium protozoan epitope as disclosed herein.

As used herein, “layer” means a thickness increment, e.g., on a template for film formation, following an adsorption step. “Multilayer” means multiple (i.e., two or more) thickness increments. A “polyelectrolyte multilayer film” is a film comprising one or more thickness increments of polyelectrolytes. After deposition, the layers of a multilayer film may not remain as discrete layers. In fact, it is possible that there is significant intermingling of species, particularly at the interfaces of the thickness increments. Intermingling, or absence thereof, can be monitored by analytical techniques such as potential measurements, X-ray photoelectron spectroscopy, and time-of-flight secondary ion mass spectrometry.

“Amino acid” means a building block of a polypeptide. As used herein, “amino acid” includes the 20 common naturally occurring L-amino acids, all other natural amino acids, all non-natural amino acids, and all amino acid mimics, e.g., peptoids.

“Naturally occurring amino acids” means glycine plus the 20 common naturally occurring L-amino acids, that is, alanine, valine, leucine, isoleucine, serine, threonine, cysteine, methionine, aspartic acid, asparagine, glutamic acid, glutamine, arginine, lysine, histidine, phenylalanine, ornithine, tyrosine, tryptophan, and proline.

“Non-natural amino acid” means an amino acid other than any of the 20 common naturally occurring L-amino acids. A non-natural amino acid can have either L- or D-stereochemistry.

“Peptoid,” or N-substituted glycine, means an analog of the corresponding amino acid monomer, with the same side chain as the corresponding amino acid but with the side chain appended to the nitrogen atom of the amino group rather than to the α-carbons of the residue. Consequently, the chemical linkages between monomers in a polypeptoid are not peptide bonds, which can be useful for limiting proteolytic digestion.

“Amino acid sequence” and “sequence” mean a contiguous length of polypeptide chain that is at least two amino acid residues long.

“Residue” means an amino acid in a polymer or oligomer; it is the residue of the amino acid monomer from which the polymer was formed. Polypeptide synthesis involves dehydration, that is, a single water molecule is “lost” on addition of the amino acid to a polypeptide chain.

As used herein “peptide” and “polypeptide” all refer to a series of amino acids connected one to the other by peptide bonds between the alpha-amino and alpha-carboxy groups of adjacent amino acids, and may contain or be free of modifications such as glycosylation, side chain oxidation, or phosphorylation, provided such modifications, or lack thereof, do not destroy immunogenicity. As used herein, the term “peptide” is meant to refer to both a peptide and a polypeptide or protein.

“Designed polypeptide” means a polypeptide that has sufficient charge for stable binding to an oppositely charged surface, that is, a polypeptide that can be deposited into a layer of a multilayer film wherein the driving force for film formation is electrostatics. In specific embodiments, a designed polypeptide is at least 15 amino acids in length and the magnitude of the net charge per residue of the polypeptide is greater than or equal to 0.1, 0.2, 0.3, 0.4 or 0.5 at pH 7.0. In one embodiment, the ratio of the number of charged residues of the same polarity minus the number of residues of the opposite polarity to the total number of residues in the polypeptide is greater than or equal to 0.5 at pH 7.0. In other words, the magnitude of the net charge per residue of the polypeptide is greater than or equal to 0.5. While there is no absolute upper limit on the length of the polypeptide, in general, designed polypeptides suitable for LBL deposition have a practical upper length limit of 1,000 residues. Designed polypeptides can include sequences found in nature such as Plasmodium protozoan epitopes as well as regions that provide functionality to the peptides such as charged regions also referred to herein as surface adsorption regions, which allow the designed polypeptides to be deposited into a polypeptide multilayer film.

“Primary structure” means the contiguous linear sequence of amino acids in a polypeptide chain, and “secondary structure” means the more or less regular types of structure in a polypeptide chain stabilized by non-covalent interactions, usually hydrogen bonds. Examples of secondary structure include α-helix, β-sheet, and β-turn.

“Polypeptide multilayer film” means a film comprising one or more designed polypeptides as defined above. For example, a polypeptide multilayer film comprises a first layer comprising a designed polypeptide and a second layer comprising a polyelectrolyte having a net charge of opposite polarity to the designed polypeptide. For example, if the first layer has a net positive charge, the second layer has a net negative charge; and if the first layer has a net negative charge, the second layer has a net positive charge. The second layer comprises another designed polypeptide or another polyelectrolyte.

“Substrate” means a solid material with a suitable surface for adsorption of polyelectrolytes from aqueous solution. The surface of a substrate can have essentially any shape, for example, planar, spherical, rod-shaped, etc. A substrate surface can be regular or irregular. A substrate can be a crystal. A substrate can be a bioactive molecule. Substrates range in size from the nanoscale to the macro-scale. Moreover, a substrate optionally comprises several small sub-particles. A substrate can be made of organic material, inorganic material, bioactive material, or a combination thereof. Nonlimiting examples of substrates include silicon wafers; charged colloidal particles, e.g., microparticles of CaCO₃ or of melamine formaldehyde; biological cells such as erythrocytes, hepatocytes, bacterial cells, or yeast cells; organic polymer lattices, e.g., polystyrene or styrene copolymer lattices; liposomes; organelles; and viruses. In one embodiment, a substrate is a medical device such as an artificial pacemaker, a cochlear implant, or a stent.

When a substrate is disintegrated or otherwise removed during or after film formation, it is called “a template” (for film formation). Template particles can be dissolved in appropriate solvents or removed by thermal treatment. If, for example, partially cross-linked melamine-formaldehyde template particles are used, the template can be disintegrated by mild chemical methods, e.g., in DMSO, or by a change in pH value. After dissolution of the template particles, hollow multilayer shells remain which are composed of alternating polyelectrolyte layers.

A “capsule” is a polyelectrolyte film in the form of a hollow shell or a coating surrounding a core. The core comprises a variety of different encapsulants, for example, a protein, a drug, or a combination thereof. Capsules with diameters less than about 1 μm are referred to as nanocapsules. Capsules with diameters greater than about 1 μm are referred to as microcapsules.

“Cross linking” means the formation of a covalent bond, or several bonds, or many bonds between two or more molecules.

“Bioactive molecule” means a molecule, macromolecule, or macromolecular assembly having a biological effect. The specific biological effect can be measured in a suitable assay and normalizing per unit weight or per molecule of the bioactive molecule. A bioactive molecule can be encapsulated, retained behind, or encapsulated within a polyelectrolyte film. Nonlimiting examples of a bioactive molecule are a drug, a crystal of a drug, a protein, a functional fragment of a protein, a complex of proteins, a lipoprotein, an oligopeptide, an oligonucleotide, a nucleic acid, a ribosome, an active therapeutic agent, a phospholipid, a polysaccharide, a lipopolysaccharide. As used herein, “bioactive molecule” further encompasses biologically active structures, such as, for example, a functional membrane fragment, a membrane structure, a virus, a pathogen, a cell, an aggregate of cells, and an organelle. Examples of a protein that can be encapsulated or retained behind a polypeptide film are hemoglobin; enzymes, such as for example glucose oxidase, urease, lysozyme and the like; extracellular matrix proteins, for example, fibronectin, laminin, vitronectin and collagen; and an antibody. Examples of a cell that can be encapsulated or retained behind a polyelectrolyte film are a transplanted islet cell, a eukaryotic cell, a bacterial cell, a plant cell, and a yeast cell.

“Biocompatible” means causing no substantial adverse health effect upon oral ingestion, topical application, transdermal application, subcutaneous injection, intramuscular injection, inhalation, implantation, or intravenous injection. For example, biocompatible films include those that do not cause a substantial immune response when in contact with the immune system of, for example, a human being.

“Immune response” means the response of the cellular or humoral immune system to the presence of a substance anywhere in the body. An immune response can be characterized in a number of ways, for example, by an increase in the bloodstream of the number of antibodies that recognize a certain antigen. Antibodies are proteins secreted by B cells, and an immunogen is an entity that elicits an immune response. The human body fights infection and inhibits reinfection by increasing the number of antibodies in the bloodstream and elsewhere.

“Antigen” means a foreign substance that elicits an immune response (e.g., the production of specific antibody molecules) when introduced into the tissues of a susceptible vertebrate organism. An antigen contains one or more epitopes. The antigen may be a pure substance, a mixture of substances (including cells or cell fragments). The term antigen includes a suitable antigenic determinant, auto-antigen, self-antigen, cross-reacting antigen, alloantigen, tolerogen, allergen, hapten, and immunogen, or parts thereof, and combinations thereof, and these terms are used interchangeably. Antigens are generally of high molecular weight and commonly are polypeptides. Antigens that elicit strong immune responses are said to be strongly immunogenic. The site on an antigen to which a complementary antibody may specifically bind is called an epitope or antigenic determinant.

“Antigenic” refers to the ability of a composition to give rise to antibodies specific to the composition or to give rise to a cell-mediated immune response. As used herein, the terms “epitope” and “antigenic determinant” are used interchangeably and mean the structure or sequence of an antigen, e.g., a peptide, which is recognized by an antibody. Ordinarily an epitope will be on the surface of a protein. A “continuous epitope” is one that involves several contiguous amino acid residues, not one that involves amino acid residues that happen to be in contact or in the limited region of space in a folded protein. A “conformational epitope” involves amino acid residues from different portions of the linear sequence of a protein that come into contact in the three-dimensional structure of the protein. For efficient interaction to occur between the antigen and the antibody, the epitope must be readily available for binding. Thus, the epitope or antigenic determinants are present in the antigen's native, cellular environment, or only exposed when denatured. In their natural form they may be cytoplasmic (soluble), membrane associated, or secreted. The number, location and size of the epitopes will depend on how much of the antigen is presented during the antibody making process.

As used herein, a “vaccine composition” is a composition which elicits an immune response in a mammal to which it is administered and which protects the immunized organism against subsequent challenge by the immunizing agent or an immunologically cross-reactive agent. Protection can be complete or partial with regard to reduction in symptoms or infection as compared with a non-vaccinated organism. An immunologically cross-reactive agent can be, for example, the whole protein (e.g., glucosyltransferase) from which a subunit peptide has been derived for use as the immunogen. Alternatively, an immunologically cross-reactive agent can be a different protein, which is recognized in whole or in part by antibodies elicited by the immunizing agent.

As used herein, an “immunogenic composition” is intended to encompass a composition that elicits an immune response in an organism to which it is administered and which may or may not protect the immunized mammal against subsequent challenge with the immunizing agent. In one embodiment, an immunogenic composition is a vaccine composition.

The invention is further illustrated by the following non-limiting examples

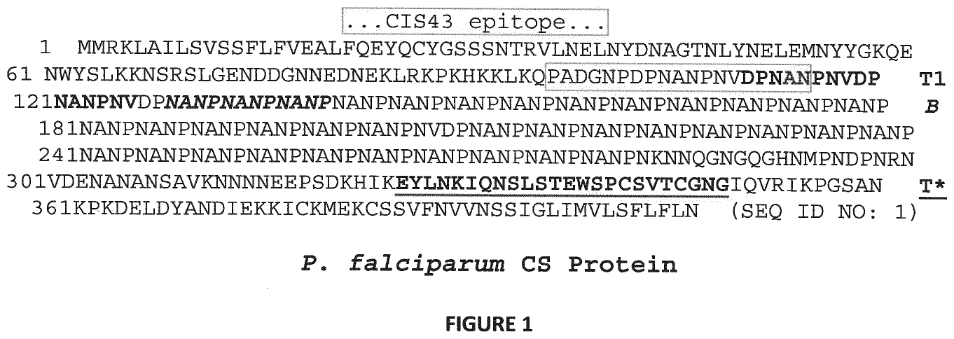

The designed peptides CIS43, T1B, T*, CIS43T1B, and CIS43T* (FIG. 1) will be synthesized and layered onto LbL-microparticles, either singly or in combination. Mice will be immunized with various permutations of vaccine design, and antibody responses will be measured by ELISA (with and without urea wash to estimate avidity) and by sporozoite neutralization assay to determine functional potency. If any formulation containing CIS43 and at least one of T1, B and T* elicits improved antibody responses, mice immunized with those formulations will be challenged with P. berghei expressing transgenic P. falciparum CS, and liver infection and parasitemia will be monitored to determine vaccine efficacy

The use of the terms “a” and “an” and “the” and similar referents (especially in the context of the following claims) are to be construed to cover both the singular and the plural, unless otherwise indicated herein or clearly contradicted by context. The terms first, second, etc., as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A composition comprising
a multilayer film comprising a plurality of oppositely charged polyelectrolyte layers,
wherein one of the polyelectrolyte layers in the multilayer film comprises an antigenic polyelectrolyte, wherein the antigenic polyelectrolyte comprises a Plasmodium falciparum circumsporozoite CIS43 epitope and one or more of T1, B, T* and T*^{M} epitopes covalently linked to the antigenic polyelectrolyte;
wherein the polyelectrolytes in the multilayer film comprise a polycationic material or a polyanionic material having a molecular weight of greater than 1,000 and at least 5 charges per molecule.

2. The composition of claim 1, wherein the antigenic polyelectrolyte comprises two of the Plasmodium falciparum circumsporozoite T1, B, T* and T*^{M} epitopes.

3. The composition of claim 1, wherein the antigenic polyelectrolyte comprises all three of the Plasmodium falciparum circumsporozoite T1, B, T* and T*^{M} epitopes.

4. The composition of claim 1, wherein the first antigenic polyelectrolyte is a polypeptide.

5. The composition of claim 1, wherein the multilayer film further comprises a TLR ligand.

6. The composition of claim 1, wherein the multilayer film is deposited on a core particle.

7. The composition of claim 6, wherein the antigenic polyelectrolyte is in the outermost layer of the multilayer film.

8. A composition comprising
a multilayer film comprising a plurality of oppositely charged polyelectrolyte layers,
wherein a first polyelectrolyte layer in the multilayer film comprises a first antigenic polyelectrolyte comprising a covalently linked Plasmodium falciparum circumsporozoite CIS43 epitope, and
wherein a second polyelectrolyte layer in the multilayer film comprises a second antigenic polyelectrolyte comprising one or more of T1, B, T* and T*^{M} epitopes covalently linked to the second polyelectrolyte, wherein the first and second polyelectrolyte layers are the same or different layers,
wherein the polyelectrolytes in the multilayer film comprise a polycationic material or a polyanionic material having a molecular weight of greater than 1,000 and at least 5 charges per molecule.

9. The composition of claim 8, wherein the second antigenic polyelectrolyte comprises two of the Plasmodium falciparum circumsporozoite T1, B, T* and T*^{M} epitopes.

10. The composition of claim 8, wherein the second antigenic polyelectrolyte comprises all three of the Plasmodium falciparum circumsporozoite T1, B, T* and T*^{M} epitopes.

11. The composition of claim 8, wherein the first antigenic polyelectrolyte, the second antigenic polyelectrolyte, or both is a polypeptide.

12. The composition of claim 8, wherein the multilayer film further comprises a TLR ligand.

13. The composition of claim 8, wherein the multilayer film is deposited on a core particle.

14. The composition of claim 8, wherein the wherein the first and second polyelectrolyte layers are in the same layer of the multilayer film.

15. The composition of claim 14, wherein the first and second polyelectrolyte layers are in the outermost layer of the multilayer film.

16. A composition comprising
a first multilayer film comprising a plurality of oppositely charged polyelectrolyte layers, wherein a first polyelectrolyte layer in the first multilayer film comprises a first antigenic polyelectrolyte comprising a covalently linked Plasmodium falciparum circumsporozoite CIS43 epitope, and
a second multilayer film comprising a plurality of oppositely charged polyelectrolyte layers, wherein a second polyelectrolyte layer in the second multilayer film comprises a second antigenic polyelectrolyte comprising one or more of T1, B, T* and T*^{M} epitopes covalently linked to the second polyelectrolyte,
wherein the polyelectrolytes in the multilayer films comprise a polycationic material or a polyanionic material having a molecular weight of greater than 1,000 and at least 5 charges per molecule.

17. The composition of claim 16, wherein the second antigenic polyelectrolyte comprises two of the Plasmodium falciparum circumsporozoite T1, B, T* and T*^{M} epitopes.

18. The composition of claim 16, wherein the second antigenic polyelectrolyte comprises all three of the Plasmodium falciparum circumsporozoite T1, B, T* and T*^{M} epitopes.

19. The composition of claim 16, wherein the first antigenic polyelectrolyte, the second antigenic polyelectrolyte, or both is a polypeptide.

20. The composition of claim 16, wherein the multilayer film further comprises a TLR ligand.

21. The composition of claim 16, wherein the first and second multilayer film is deposited on separate core particles.

22. A method of eliciting an immune response in a vertebrate organism comprising administering into the vertebrate organism the composition of claim 1.

23. A method of eliciting an immune response in a vertebrate organism comprising administering into the vertebrate organism the composition of claim 8.

24. A method of eliciting an immune response in a vertebrate organism comprising administering into the vertebrate organism the composition of claim 16.


SEQUENCE LISTING (21 sequences)

SEQ ID NO: 1 - protein, 397 aa, Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 2 - protein, 16 aa, plasmodium falciparum
DPNANPNVDPNANPNV

SEQ ID NO: 3 - protein, 4 aa, plasmodium falciparum
NANP

SEQ ID NO: 4 - protein, 20 aa, plasmodium falciparum
EYLNKIQNSLSTEWSPCSVT

SEQ ID NO: 5 - protein, 20 aa, artificial
  modified t* epitope
EYLNKIQNSLSTEWSPSSVT

SEQ ID NO: 6 - protein, 15 aa, plasmodium falciparum
PADGNPDPNANPNVS

SEQ ID NO: 7 - protein, 91 aa, artificial
  Designed peptide T1BT*M
DESIGNEDPEPTIDETBTMDPNANPNVDPNANPNVNANPNANPNANPEYLNKIQNSLSTEWSPSSVTSGNGKKKKKKKKKKKKKKKKKKKK

SEQ ID NO: 8 - protein, 72 aa, ARTIFICIAL
  Designed peptide T1BT*
DPNANPNVDPNANPNVNANPNANPNANPEYLNKIQNSLSTEWSPCSVTSGNGKKKKKKKKKKKKKKKKKKKK

SEQ ID NO: 9 - protein, 72 aa, ARTIFICIAL
  Designed peptide T*M
DPNANPNVDPNANPNVNANPNANPNANPEYLNKIQNSLSTEWSPSSVTSGNGKKKKKKKKKKKKKKKKKKKK

SEQ ID NO: 10 - protein, 72 aa, ARTIFICIAL; identical to SEQ ID NO: 8
  Designed peptide T*

SEQ ID NO: 11 - protein, 48 aa, ARTIFICIAL
  Designed peptide T1B
DPNANPNVDPNANPNVNANPNANPNANPKKKKKKKKKKKKKKKKKKKK

SEQ ID NO: 12 - protein, 39 aa, ARTIFICIAL
  peptide CIS43
PADGNPDPNANPNVDPNANKKKKKKKKKKKKKKKKKKKK

SEQ ID NO: 13 - protein, 86 aa, ARTIFICIAL
  Designed peptide CIS43T1BT*M
PADGNPDPNANPNVDPNANPNVDPNANPNVNANPNANPNANPEYLNKIQNSLSTEWSPSSVTSGNGKKKKKKKKKKKKKKKKKKKK

SEQ ID NO: 14 - protein, 86 aa, ARTIFICIAL
  Designed peptide CIS43T1BT*
PADGNPDPNANPNVDPNANPNVDPNANPNVNANPNANPNANPEYLNKIQNSLSTEWSPCSVTSGNGKKKKKKKKKKKKKKKKKKKK

SEQ ID NO: 15 - protein, 62 aa, ARTIFICIAL
  Designed peptide CIS43T1B
PADGNPDPNANPNVDPNANPNVDPNANPNVNANPNANPNANPKKKKKKKKKKKKKKKKKKKK

SEQ ID NO: 16 - protein, 62 aa, ARTIFICIAL; identical to SEQ ID NO: 15
  Designed peptide CIS43T1

SEQ ID NO: 17 - protein, 51 aa, ARTIFICIAL
  Designed peptide CIS43B
PADGNPDPNANPNVDPNANNANPNANPNANPKKKKKKKKKKKKKKKKKKKK

SEQ ID NO: 18 - protein, 63 aa, ARTIFICIAL
  Designed peptide CIS43T*M
PADGNPDPNANPNVDPNANEYLNKIQNSLSTEWSPSSVTSGNGKKKKKKKKKKKKKKKKKKKK

SEQ ID NO: 19 - protein, 63 aa, ARTIFICIAL
  Designed peptide CIS43T*
PADGNPDPNANPNVDPNANEYLNKIQNSLSTEWSPCSVTSGNGKKKKKKKKKKKKKKKKKKKK

SEQ ID NO: 20 - protein, 15 aa, plasmodium falciparum
NPDPNANPNVDPNAN

SEQ ID NO: 21 - protein, 15 aa, plasmodium falciparum
NANPNANPNANPNAN